(19) United States
(12) Patent Application Publication
(10) Pub. No.: US 2004/0259189 A1
(43) Pub. Date: Dec. 23, 2004

(54) Water supply monitoring for contaminant detection

(76) Inventor: Carl R. Marschke, Phillips, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 10/456,643

(22) Filed: Jun. 6, 2003

Related U.S. Application Data
(60) Provisional application No. 60/387,722, filed on Jun. 7, 2002.

(51) Int. Cl. C12Q 1/04

(52) U.S. Cl. 435/34

(57) ABSTRACT

A system for monitoring and detecting the presence of hazardous material in a water supply system includes monitoring stations distributed throughout the water supply system for detecting the presence of hazardous materials injected downstream of the water treatment source. The system may also include a sampling device for capturing a water sample in response to detection of a hazardous material for more precise analysis.

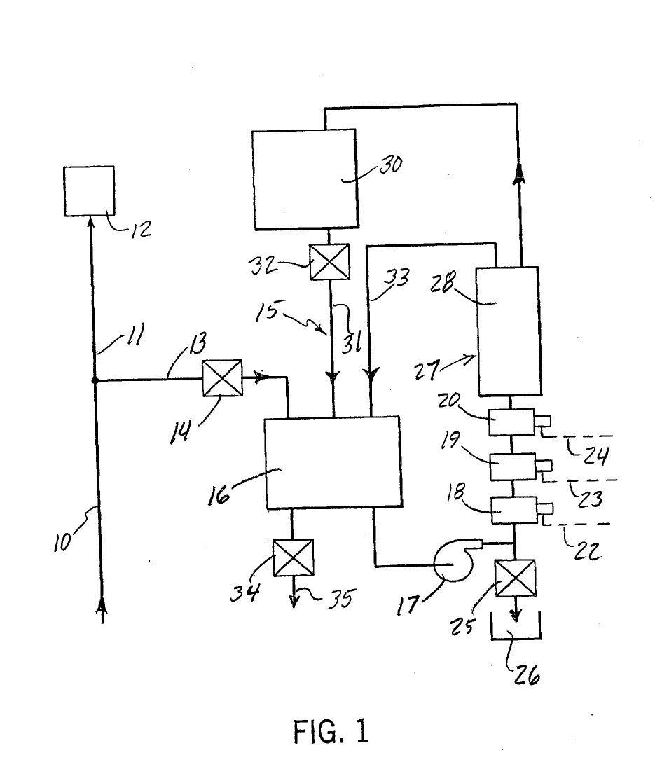

### CROSS REFERENCE TO RELATED APPLICATION

The present invention is based on U.S. Provisional Patent Application Ser. No. 60/387,722, filed on Jun. 7, 2002.

### BACKGROUND OF THE INVENTION

The present invention pertains to a system for monitoring a water supply system on a continuous basis to detect the presence of hazardous materials injected into the system downstream from the water treatment station. More particularly, the invention pertains to a system disposed on a distributed basis at critical points throughout an entire water supply system to increase the chances that remotely injected hazardous materials would be detected as soon as possible to permit the system to be shutdown.

Based on the construction and layout of most municipal water systems, a branched network of mains distributes water throughout a municipality from one or more treatment stations. Typically, untreated water from a source is screened and filtered, as with a sand filter, followed by chlorination before entering the distribution system. The branched system of mains typically feeds laterals to buildings, factories, residences or other centralized user locations. A lateral to a user location will normally include a shutoff valve followed by a water meter. The meter may have valves on either side to isolate it from the system as for repair or replacement.

It is also known to utilize vacuum breakers in the system within a user location to prevent the back flow of water into the supply system in the event of loss of system supply pressure. However, there are typically no check valves in the laterals to absolutely prevent back flow in higher latitudes where freezing is a problem. In lower latitudes, the back flow preventers consist of double check valves with a shut-off valve on each side. These are typically located above ground at the street or inside the building ahead of the meter. In either case, these systems could be easily defeated by replacing the check valves with valves that have the internal parts removed with no change to the external appearance.

In view of the foregoing considerations, it would be a relatively simple and straightforward operation for a terrorist or other person with evil intent to tap into a water supply lateral and, with a high pressure and high volume pump operating at greater than the water supply pressure, to pump a hazardous substance into the system. The hazardous substance could be biological, chemical or radioactive. Furthermore, the equipment needed and even many kinds of hazardous chemicals can be purchased or obtained without arousing suspicion, and installed at a private, i.e. owned or leased, location.

Because of the manner in which typical drinking water supply systems are constructed and operated, it would be virtually impossible to prevent the foregoing type of terrorist activity, the results of which could be devastating in any water supply system. However, in accordance with the present invention a distributed system of monitoring stations could be utilized to detect the infiltration into the water supply system of a hazardous substance and to generate an appropriate signal to have the system shut down as quickly as possible.

### SUMMARY OF THE INVENTION

In accordance with the present invention, monitoring systems, capable of monitoring for and detecting biological, chemical or radioactive hazards, are distributed broadly throughout a water supply system, and operated to monitor for the infiltration of any one of these hazards on a continuous basis. The monitoring and detection systems must be distributed throughout the system because a hazardous material could be injected virtually anywhere into the system. In this manner and only in this manner could the appearance of a hazardous substance in a water supply system be detected quickly enough to prevent catastrophic results.

Detection methods and apparatus already exist for chemical detection, such as spectral analyses. The detection of selected biological substances is also known using, for example, biochemical assay systems as disclosed in U.S. Pat. No. 6,051,388 or liquid crystal detectors. Radiation detectors are also available. Information has also recently been published concerning the development of a DNA chip that can detect the presence of water borne pathogens, such as cryptosporidium, in a water supply. Similarly, the development of an optical bio-chip has recently been reported which provides the potential for detecting harmful biological and chemical agents introduced into a water supply. Other optical sensor technology has also been applied in the development of miniaturized detection systems for both radioactive and chemical contaminants in which the natural response of bacterial cells to such contaminants are detected with optical sensors.

In a distributed system as contemplated by the subject invention, each location in the distributed network would have the capability of monitoring for and detecting all three types of hazards, namely, chemical, biological and radioactive. Each detector system would be installed in a location where continuous flow of water could be monitored. Furthermore, the widely distributed system would likely have to include privately owned and operated monitoring and detection systems, since an effective system would by necessity have to include locations under private ownership. Nevertheless, all of the monitoring stations could be tied into a communication system that would immediately alert the appropriate government authorities to take action to shut the system down.

In a specific instance of detection of a hazardous substance or condition, signal from the detecting station might be used to isolate and allow shut down of a specific branch or portion of the water distribution system, while the distributed monitoring system continues to operate.

In conjunction with the monitoring and detection system of the present invention, it would be desirable to include a subsystem which would capture and store a sample immediately upon detection of a contaminant. The sample could then be utilized for more precise analysis. For example, an automated sampling device connected to the water supply system and utilizing an evacuated container into which a sample of water containing a detected contaminant would be captured. The system could utilize, for example, a needle penetrating a seal to permit entry of the water sample into the container to assure a sterile sample.

### DETAILED DESCRIPTION OF THE PREFERRED EMBODIMENT

In the single drawing figure, there is shown a schematic representation of the monitoring system of the present invention, including a subsystem for periodic sampling, concentrating the sample, if necessary, and capturing a positive sample for more precise analysis. A treated water supply line 10 may be, for example, a lateral 11 from a main (not shown) supplying water to a building 12. The lateral 11 is tapped to provide a sampling line 13 to the detection system 15, including a shut-off valve 14.

The detection system inlet comprises a sample reservoir 16 which, under the control of the primary shut-off valve 14, takes a periodic sample from the supply line 10. Samples might be taken, for example, at 10 minute intervals. From the sample reservoir 16, the water is pumped via pump 17 through a series of detectors, including a chemical detector 18, a biological detector 19, and a radiation detector 20. If a raw water sample from the sample reservoir 16 includes a contaminant or hazardous material within the detection range of a sensor used in one of the detectors 18-20, an immediate alarm signal may be generated via connection lines 22-24, respectively, to a central control or other alarm-generating device. Once a positive indication of the presence of hazardous material in the water is generated, a sample valve 25 is opened to capture a sample in a test sample chamber 26.

However, if the contaminant or other hazardous material in the water supply is at a concentration too low for initial detection, a concentrator 27 may be utilized to concentrate the sample in the sample reservoir 16 for recirculation through the detectors. Downstream from the radiation detector 20, a reverse osmosis (RO) device utilizes a conventional semi-permeable membrane to separate the sample into essentially pure water permeate and a concentrate containing all of the material retained on the membrane. The pure water permeate is directed to a pure water reservoir 30 which includes a pure water return line 31 selectively isolated from the sample reservoir 16 with a pure water valve 32. The concentrate flows continuously through a concentrate line 33 into the sample reservoir 16. During the sample period, the pump 17 continues to circulate the concentrate until the contaminant is at a level sufficient for detection. When the detection level is reached, a signal will be generated by one of the respective detectors 18-20. As indicated previously, the sample valve 25 will open and a sample will be taken by the sample chamber 26. This sample may then be utilized for more precise analysis. However, an appropriate alarm signal will already have been generated by one of the detectors 18, 19 or 20.

Once a sample has been concentrated and tested, some of the permeate in the pure water reservoir 30 may be used to rinse the concentrated sample from the sample reservoir 16 and drain it from the sample reservoir 16 via a drain valve 34 and drain line 35. Pure water remaining in the reservoir 30 may then be discharged in stages to the sample reservoir 16, circulated by the pump 17 through the detectors and the concentrator 27 to rinse the system.

1. In a water supply system including a plurality of conduits comprising a network of main water supply lines feeding laterals that deliver water from a treatment source to discrete user locations served by a lateral, a monitoring system for detecting the presence in the supply system of hazardous contaminant injected downstream of the treatment source, said monitoring system comprising a series of monitoring stations distributed broadly throughout the entire water supply system network, each station including direct access to a continuous flow of water, and means for detecting the presence of multiple types of hazardous contaminants; and,
means for transmitting a signal based on detection of a contaminants to enable all or a portion of the system to be shut down as quickly as possible regardless of where in the system the contaminant is injected.

2. The monitoring system as set forth in claim 1 including means for concentrating the contaminant in the flow of water to a given detection concentration level.

3. In a water supply system including a plurality of mains and laterals comprising a network of water supply lines delivering water from a treatment source to discrete user locations served by laterals, a monitoring system for detecting the presence in the supply system of hazardous material injected downstream of the treatment source, said monitoring system comprising a series of monitoring stations distributed broadly throughout the entire water supply system network, each station including direct access to a continuous flow of water, and detectors for detecting the presence of multiple types of hazardous materials; and,
a communication link connecting each station to a central control to transmit a signal based on detection of a hazardous material to enable al or a portion of the system to be shut down immediately regardless of where in the system the hazardous material is injected.

4. The monitoring system as set forth in claim 3 wherein said hazardous materials are selected from the group consisting of chemical, biological and radioactive contaminants.

5. The system as set forth n claim 3 including a sampling device for capturing a water sample in response to detection of a hazardous material.

6. A method for monitoring a water supply system including a network of main and lateral water supply lines delivering water from a treatment source to detect the presence of hazardous material injected downstream of the treatment source, said method comprising the steps of:
(1) broadly distributing a series of monitoring stations in supply lines throughout the water supply system;
(2) providing direct access to a continuous flow of water in the system at each of said monitoring stations;
(3) operating the monitoring stations to detect the presence of a hazardous material in the water supply system;
(4) transmitting a signal based on detection of a hazardous material and
(5) utilizing the signal to enable authorities to take immediate action including shutting down all or a portion of the system.

7. A method as set forth in claim 6 including the step of capturing a water sample in response to detection of a hazardous material.
